(19) United States
(12) Patent Application Publication
(10) Pub. No.: US 2015/0258072 A1
(43) Pub. Date: Sep. 17, 2015

(54) Antibacterial compositions

(71) Applicant: WOCKHARDT LIMITED, Chikalthana, Aurangabad (IN)

(72) Inventors: Sachin Bhagwat, Aurangabad (IN); Mahesh Vithalbhai Patel, Aurangabad 3 (IN)

(21) Appl. No.: 14/433,883

(22) PCT Filed: May 13, 2013
(86) PCT No.: PCT/IB2013/053867

§ 371 Date: Apr. 7, 2015

(30) Foreign Application Priority Data
Sep. 3, 2012 (IN) 2552/MUM/2012

(51) Int. Cl. A61K 31/431; A61K 31/424; A61K 31/546; A61K 31/43

(52) U.S. Cl. CPC A61K 31/431; A61K 31/43; A61K 31/424; A61K 31/546

(57) ABSTRACT

A pharmaceutical composition useful for the treatment or control of bacterial infections is disclosed.

### FIELD OF THE INVENTION

The invention relates to antibacterial compositions and methods of treating or controlling bacterial infections.

### BACKGROUND OF THE INVENTION

The accelerated emergence of antibiotic resistance among the widely prevalent pathogens is the most serious threat to the management of infectious diseases. For Gram negatives, resistance to beta-lactam, fluoroquinolone and aminoglycoside class of antibiotic agents is currently posing a considerable challenge to clinicians. Beta-lactam antibiotics have historically remained the mainstay of therapy for the management of wide range of serious Gram-negative infections.

In general, there are four primary mechanisms by which bacteria can overcome beta-lactam antibiotic agents. These mechanisms include production of beta-lactamases, decreased expression of outer membrane proteins, efflux pumps and mutations in the active site of Penicillin Binding Proteins (PBPs). Production of beta-lactamases is the most dominant mechanism of resistance to this class of antibiotic. Introduction of extended spectrum beta-lactam antibiotic agents during 1980s were responded by Gram negative organisms with the production of Extended Spectrum Beta-lactamases (ESBLs). The ESBLs are heterogenous group of plasmid mediated enzymes, now numbering more than 890, (Bush et al, Critical Care 2010; 14:224) imparting various degrees of resistance to broader spectrum of beta-lactam antibiotic agents including third and fourth generation cephalosporins.

ESBL producing Enterobacteriaceae are causing a worldwide epidemic of urinary tract infections in community and inpatient settings as well as severe infections in hospital populations. Their prevalence varies from one country to another and from institution to institution. E. coli and Klebsiella account for the majority of the pathogens expressing ESBLs in most parts of the world. AmpC hyperproducing E. coli and Klebsiella are recognized worldwide as important nosocomial pathogens and have also been associated with hospital acquired urinary tract infections, blood stream infections and other severe infections such as intra-abdominal infections and sepsis.

Given the ability of ESBL producing organisms to hydrolyze several beta-lactam antibiotic agents, it is not surprising that antibiotic choice for infections with such organisms is seriously reduced. Furthermore, plasmid-bearing genes encoding ESBLs frequently also carry genes encoding resistance to quinolones, aminoglycosides and trimethoprim-sulphamethaxozole. Invariably, injectable agents such as carbapenems are employed since there are no effective oral options available. Current oral options such as quinolones and oral cephalosporins are ineffective in tackling quinolone and ESBL resistance respectively which coexists in many clinical isolates. Therefore ESBL including Class A and C effective oral options are urgently needed.

### SUMMARY OF THE INVENTION

Accordingly, there are provided pharmaceutical compositions and methods for the treatment or control of bacterial infections.

In one general aspect, there are provided pharmaceutical compositions useful for the treatment or control of bacterial infections, the composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In another general aspect, there are provided methods for the treatment or control of bacterial infections, said method comprising administering to a patient in need thereof, effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In yet another general aspect, there are provided methods for the treatment or control of bacterial infections, said method comprising administering to a patient in need thereof, a pharmaceutical composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In another general aspect, there are provided methods for increasing antibiotic effectiveness of a beta-lactam antibiotic agent in a subject, said method comprising co-administering said beta-lactam antibiotic agent with effective amounts of: (a) at least one beta-lactamase inhibitor or a pharmaceutically acceptable derivative thereof, and (b) mecillinam or a pharmaceutically acceptable derivative thereof, wherein the beta-lactam antibiotic agent is not mecillinam or a pharmaceutically acceptable derivative thereof.

The details of one or more embodiments of the invention are set forth in the description below. Other features, objects and advantages of the inventions will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It must be noted that, as used in this specification and the appended claims, the singular forms “a,” “an,” and “the” include plural referents unless the content clearly dictates otherwise.

The inventors have surprisingly discovered that a pharmaceutical composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof, exhibits unexpectedly improved and synergistic antibacterial efficacy, even against highly resistant ESBL producing bacteria.

The term “infection” as used herein includes presence of microbes, including bacteria, in or on a subject, which, if its growth were inhibited, would result in a benefit to the subject. As such, the term “infection” in addition to referring to the presence of bacteria also refers to normal flora which, are not desirable. The term “infection” includes infection caused by bacteria.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection. The term “therapeutic treatment” refers to administering treatment to a subject already suffering from infection. The term “treat”, “treating” or “treatment” as used herein also refers to administering compositions or one or more of pharmaceutically active ingredients discussed herein, with or without additional pharmaceutically active or inert ingredients, in order to: (i) reduce or eliminate either a bacterial infection or one or more symptoms of the bacterial infection, or (ii) retard the progression of a bacterial infection or of one or more symptoms of the bacterial infection, or (iii) reduce the severity of a bacterial infection or of one or more symptoms of the bacterial infections, or (iv) suppress the clinical manifestation of a bacterial infection, or (v) suppress the manifestation of adverse symptoms of the bacterial infections.

The term “control” or “controlling” as used herein generally refers to preventing, reducing, or eradicating infection or inhibiting the rate and extent of such infection, or reducing the microbial population, such as a microbial population present in or on a body or structure, surface, liquid, subject, etc, wherein such prevention or reduction in the infection or microbial population is statistically significant with respect to untreated infection or population. In general, such control may be achieved by increased mortality amongst the microbial population.

The term “effective amount” as used herein refers to an amount, which has a therapeutic effect or is the amount required to produce a therapeutic effect in a subject. For example, a therapeutically or pharmaceutically effective amount of an antibiotic agent or a pharmaceutical composition is the amount of the antibiotic agent or the pharmaceutical composition required to produce a desired therapeutic effect as may be judged by clinical trial results, model animal infection studies, and/or in vitro studies (e.g. in agar or broth media). The effective or pharmaceutically effective amount depends on several factors, including but not limited to, the microorganism (e.g. bacteria) involved, characteristics of the subject (for example height, weight, sex, age and medical history), severity of infection and the particular type of the antibiotic used. For prophylactic treatments, a therapeutically or prophylactically effective amount is that amount which would be effective to prevent a microbial (e.g. bacterial) infection.

The term “administration” or “administering” includes delivery of a composition or one or more pharmaceutically active ingredients to a subject, including for example, by any appropriate methods, which serves to deliver the composition or it's active ingredients or other pharmaceutically active ingredients to the site of the infection. The method of administration can vary depending on various factors, such as for example, the components of the pharmaceutical composition or the typenature of the pharmaceutically active or inert ingredients, the site of the potential or actual infection, the microorganism involved, severity of the infection, age and physical condition of the subject. Some non-limiting examples of ways to administer a composition or a pharmaceutically active ingredient to a subject according to this invention includes oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In case of a pharmaceutical composition comprising more than one ingredients (active or inert), one of way of administering such composition is by admixing the ingredients (e.g. in the form of a suitable unit dosage form such as tablet, capsule, solution, powder etc.) and then administering the dosage form. Alternatively, the ingredients may also be administered separately (simultaneously or one after the other) as long as these ingredients reach beneficial therapeutic levels such that the composition as a whole provides a synergistic effect.

The term “growth” as used herein refers to a growth of microorganisms and includes reproduction or population expansion of the microorganism (e.g. bacteria). The term also includes maintenance of on-going metabolic processes of a microorganism, including processes that keep the microorganism alive.

The term, “effectiveness” as used herein refers to ability of a treatment or a composition or one or more pharmaceutically active ingredients to produce a desired biological effect in a subject. For example, the term “antibiotic effectiveness” of a composition or a beta-lactam antibiotic agent refers to the ability of the composition or the beta-lactam antibiotic agent to prevent or treat the microbial (e.g. bacterial) infection in a subject.

The term “synergistic” or “synergy” as used herein refers to the interaction of two or more agents so that their combined effect is greater than their individual effects.

The term ““pharmaceutically inert ingredient” or “carrier” or “excipient” refers to a compound or material used to facilitate administration of a compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as oil, peanut and sesame oils. In addition, various adjuvants commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The term “antibiotic agent” as used herein refers to any substance, compound or a combination of substances or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibiotic agent” also refers to compounds capable of decreasing infectivity or virulence of bacteria.

The term “pharmaceutically acceptable derivative” as used herein is meant to include any pharmaceutically acceptable salt, pro-drugs, metabolites, esters, ethers, hydrates, polymorphs, solvates, complexes, enantiomers or adducts of a compound of the present invention which, upon administration to the subject, is capable of providing (directly or indirectly) the parent compound.

In general, the pharmaceutically acceptable salts are well known in the art. For example, S. M. Berge, et al. (J. Pharmaceutical Sciences, 66: 1-19 (1977)), incorporated herein by reference, describes various pharmaceutically acceptable salts in details.

The term “subject” as used herein refers to vertebrate or invertebrate, including a mammal. The term “subject” includes human, animal, a bird, a fish, or an amphibian. Typical, non-limiting examples of a “subject” includes humans, cats, dogs, horses, sheep, bovine cows, pigs, lambs, rats, mice and guinea pigs.

The term “beta-lactam antibiotic agent” as used herein refers to compounds with antibiotic properties and containing a beta-lactam nucleus in their molecular structure.

The term “beta-lactamase” as used herein refers to any enzyme or protein or any other substance that breaks down a beta-lactam ring. The term “beta-lactamase” includes enzymes that are produced by bacteria and have the ability to hydrolyze the beta-lactam ring in a beta-lactam antibiotic, either partially or completely.

The term “beta-lactamase inhibitor” as used herein refers to a compound capable of inhibiting activity of one or more beta-lactamase enzymes, either partially or completely.

A person of skills in the art would appreciate that various compounds described herein (including, for example, the beta-lactam antibiotic and the beta-lactamase inhibitor) can exist and are often used as their pharmaceutically acceptable derivatives such as salts, pro-drugs, metabolites, esters, ethers, hydrates, polymorphs, solvates, complexes, enantiomers, adducts or other pharmaceutically acceptable derivatives. A reference to compounds discussed herein, therefore, is intended to include such pharmaceutically acceptable salts, pro-drugs, metabolites, esters, ethers, hydrates, polymorphs, solvates, complexes, enantiomers, adducts or their any other pharmaceutically acceptable derivatives. For example, the terms “beta-lactam antibiotic”, and “beta-lactamase inhibitor” includes their pharmaceutically acceptable salts, pro-drugs, metabolites, esters, ethers, hydrates, polymorphs, solvates, complexes, enantiomers, adducts or their any other pharmaceutically acceptable derivatives.

In one general aspect, there are provided pharmaceutical composition useful for the treatment or control of bacterial infections, the composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In another general aspect, there are provided methods for the treatment or control of bacterial infections, said method comprising administering to a patient in need thereof, effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In another general aspect, there are provided methods for the treatment or control of bacterial infections, said method comprising administering to a patient in need thereof, a pharmaceutical composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

In a further general aspect, there are provided methods for increasing antibiotic effectiveness of a beta-lactam antibiotic agent in a subject, said method comprising co-administering said beta-lactam antibiotic agent with effective amounts of: (a) at least one beta-lactamase inhibitor or a pharmaceutically acceptable derivative thereof, and (b) mecillinam or a pharmaceutically acceptable derivative thereof, wherein the beta-lactam antibiotic agent is not mecillinam or a pharmaceutically acceptable derivative thereof.

The compositions and methods according to the invention include at least one beta-lactam antibiotic agent. In some embodiments, the beta-lactam antibiotic agent is selected from the group consisting of penicillins, penems, carbapenems, cephems, and monobactams.

In some other embodiments, the beta-lactam antibiotic agent is a penicillin compound. Typical, non-limiting examples of penicillin compounds include amoxicillin, ampicillin, pivampicillin, hetacillin, bacampicillin, metampicillin, talampicillin, epicillin, carbenicillin (carindacillin), ticarcillin, temocillin, azlocillin, piperacillin, mezlocillin, sulbenicillin, benzylpenicillin (G), clometocillin, benzathine benzylpenicillin, azidocillin, penamecillin, phenoxymethylpenicillin (V), propicillin, benzathine phenoxymethylpenicillin, pheneticillin, cloxacillin, dicloxacillin, flucloxacillin, oxacillin, meticillin, nafcillin or a pharmaceutically acceptable derivative thereof.

In some other embodiments, the beta-lactam antibiotic agent is a penem compound. Typical, non-limiting examples of penem compounds include faropenem or a pharmaceutically acceptable derivative thereof.

In some embodiments, the beta-lactam antibiotic agent is a carbapenem compound. Typical, non-limiting examples of carbapenem compounds include the biapenem, ertapenem, doripenem, imipenem, meropenem, panipenem, or a pharmaceutically acceptable derivative thereof.

In some embodiments, the beta-lactam antibiotic agent is a cephem compound. In general, the cephem compounds include cephalosporins, cephamycins, and carbacephems. Typical, non-limiting examples of cephem compounds include cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cefoxitin, cefotetan, cefmetazole, loracarbef, cefixime, ceftriaxone, ceftazidime, cefoperazone, cefcapene, cefdaloxime, cefdinir, cefditoren, cefetamet, cefmenoxime, cefodizime, cefotaxime, cefpimizole, cefpiramide, cefpodoxime, cefsulodin, cefteram, ceftibuten, ceftiolene, ceftizoxime, flomoxef, latamoxef, cefepime, cefozopran, cefpirome, cefquinome, ceftobiprole, ceftaroline fosamil, ceftiofur, cefquinome, cefovecin or a pharmaceutically acceptable derivative thereof.

In some other embodiments, the beta-lactam antibiotic agent is a monobactam compound. Typical, non-limiting examples of monobactam compounds include aztreonam, tigemonam, carumonam, nocardicin A, or a pharmaceutically acceptable derivative thereof.

In some embodiments, the beta-lactam antibiotic agent is amoxicillin or a pharmaceutically acceptable derivative thereof. In some other embodiments, the beta-lactam antibiotic is cefixime, cefuroxime, cefpodoxime, cefaclor, cefprozil, cefdinir, cefditoren or a pharmaceutically acceptable derivative thereof.

The compositions and methods according to the invention include a beta-lactamase inhibitor. Typical, non-limiting examples of such beta-lactamase inhibitors include sulbactam, tazobactam, clavulanic acid, or a pharmaceutically acceptable derivative thereof. In some embodiments, the beta-lactamase inhibitor is clavulanic acid or its pharmaceutically acceptable salt (for example, potassium or a sodium salt).

In some embodiment, mecillinam is present as pivmecillinam (also known as mecillinam pivoxil).

The amount of each component in the composition and methods according to the invention can vary widely depending on the requirements.

In some embodiments, the beta-lactam antibiotic agent or a pharmaceutically acceptable derivative thereof in the compositions and methods according to invention is present in an amount of about 0.1 mg to about 3000 mg.

In some other embodiments, the beta-lactamase inhibitor or a pharmaceutically acceptable derivative thereof in the composition and methods according to the invention is present in an amount of about 0.1 mg to about 3000 mg.

In some embodiments, mecillinam or a pharmaceutically acceptable derivative thereof in the composition and methods according to the invention is present in an amount of about 0.1 mg to about 3000 mg.

In some embodiments, there is provided a pharmaceutical composition useful for the treatment or control of bacterial infections, the composition comprising effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, a pharmaceutical composition comprising effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

In some embodiments, amoxicillin or a pharmaceutically acceptable derivative thereof in the compositions and methods according to invention is present in an amount of about 0.1 mg to about 3000 mg.

In some other embodiments, clavulanic acid or a pharmaceutically acceptable derivative thereof in the composition and methods according to the invention is present in an amount of about 0.1 mg to about 3000 mg.

In some embodiments, mecillinam or a pharmaceutically acceptable derivative thereof in the composition and methods according to the invention is present in an amount of about 0.1 mg to about 3000 mg.

In some embodiments, there is provided a pharmaceutical composition useful for the treatment or control of bacterial infections, the composition comprising effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, a pharmaceutical composition comprising effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

In some embodiments, cefixime or a pharmaceutically acceptable derivative thereof in the compositions and methods according to invention is present in an amount of about 0.1 mg to about 3000 mg.

In some other embodiments, clavulanic acid or a pharmaceutically acceptable derivative thereof in the composition and methods according to the invention is present in an amount of about 0.1 mg to about 3000 mg.

In some embodiments, mecillinam or a pharmaceutically acceptable derivative thereof in the composition and methods according to the invention is present in an amount of about 0.1 mg to about 3000 mg.

In some embodiments, there is provided a pharmaceutical composition useful for the treatment or control of bacterial infections, the composition comprising effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, a pharmaceutical composition comprising effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

In some embodiments, cefuroxime or a pharmaceutically acceptable derivative thereof in the compositions and methods according to invention is present in an amount of about 0.1 mg to about 3000 mg.

In some other embodiments, clavulanic acid or a pharmaceutically acceptable derivative thereof in the composition and methods according to the invention is present in an amount of about 0.1 mg to about 3000 mg.

In some embodiments, mecillinam or a pharmaceutically acceptable derivative thereof in the composition and methods according to the invention is present in an amount of about 0.1 mg to about 3000 mg.

In some embodiments, there is provided a pharmaceutical composition useful for the treatment or control of bacterial infections, the composition comprising effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.

In some other embodiments, there is provided a method for the treatment or control of bacterial infections, said method comprising administering to a patient in need thereof, a pharmaceutical composition comprising effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.

In some embodiments, cefpodoxime or a pharmaceutically acceptable derivative thereof in the compositions and methods according to invention is present in an amount of about 0.1 mg to about 3000 mg.

In some other embodiments, clavulanic acid or a pharmaceutically acceptable derivative thereof in the composition and methods according to the invention is present in an amount of about 0.1 mg to about 3000 mg.

In some embodiments, mecillinam or a pharmaceutically acceptable derivative thereof in the composition and methods according to the invention is present in an amount of about 0.1 mg to about 3000 mg.

In the methods, the pharmaceutical composition and/or other pharmaceutically active ingredients according to the invention may be administered by any appropriate method, which serves to deliver the composition or its constituents or the active ingredients to the desired site. The method of administration can vary depending on various factors, such as for example, the components of the pharmaceutical composition and nature of the active ingredients, the site of the potential or actual infection, the microorganism (e.g. bacteria) involved, severity of infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash.

In some embodiments, the compositions or active ingredients thereof are administered orally.

The compositions according to the invention can be formulated into various dosage forms wherein the active ingredients may be present either together (e.g. as an admixture) or as separate components. When the various ingredients in the composition are formulated as a mixture, such composition can be delivered by administering such a mixture. The composition or dosage form wherein the ingredients do not come as a mixture, but come as separate components, such composition dosage form can be administered in several ways. In one possible way, the ingredients can be mixed in the desired proportions and the mixture is then administered as required. Alternatively, the components can be separately administered in appropriate proportions so as to achieve the same or equivalent therapeutic level or effect as would have been achieved by administration of the equivalent mixture.

Similarly, in the methods according to the invention, the active ingredients can be administered to a subject in several ways depending on the requirements. In some embodiments, the active ingredients are admixed in appropriate amounts and then the admixture is administered to a subject. In some other embodiments, the active ingredients are administered separately. Since the invention contemplates that the active ingredients agents may be administered separately, the invention further provides for combining separate pharmaceutical compositions in kit form. The kit may comprise one or more separate pharmaceutical compositions, each comprising one or more active ingredients. Each of such separate compositions may be present in a separate container such as a bottle, vial, syringes, boxes, bags, and the like. Typically, the kit comprises directions for the administration of the separate components. The kit form is particularly advantageous when the separate components are preferably administered in different dosage forms (e.g., oral and parenteral) ore are administered at different dosage intervals. When the active ingredients are administered separately, they may be administered simultaneously or sequentially.

The pharmaceutical composition or the active ingredients according to the present invention may be formulated into a variety of dosage forms. Typical, non-limiting examples of dosage forms include solid, semi-solid, liquid and aerosol dosage forms; such as tablets, capsules, powders, solutions, suspensions, suppositories, aerosols, granules, emulsions, syrups, elixirs and a like.

In general, the pharmaceutical compositions and method disclosed herein are useful in treating or controlling bacterial infections. Advantageously, the compositions and methods disclosed herein are particularly effective in preventing or treating infections caused by bacteria that are considered be less or not susceptible to one or more of known beta-lactam antibiotic or their known compositions. Some non-limiting examples of such bacteria known to have developed resistance to various antibacterial agents include Acinetobacter, E. coli, Pseudomonas aeruginosa, Staphylococcus aureus, Enterobacter, Klebsiella, Citrobacter and a like. Other non-limiting examples of infections that may be prevented or treated using the compositions and/or methods of the invention include: skin and soft tissue infections, febrile neutropenia, urinary tract infection, intraabdominal infections, respiratory tract infections, pneumonia (nosocomial), bacteremia meningitis, surgical, infections etc.

Surprisingly, the compositions and methods according to the invention are also effective in treating or controlling bacterial infections that are caused by bacteria producing one or more beta-lactamase enzymes. The ability of compositions and methods according to the present invention to treat such resistant with typical beta-lactam antibiotics represents a significant improvement in the art.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions. All references including patents, patent applications, and literature cited in the specification are expressly incorporated herein by reference in their entirety.

### Examples

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

The efficacy of compositions and methods in treating or controlling bacterial infections was studied. In a typical study, overnight grown bacterial cultures were diluted appropriately and inoculated on the agar media containing doubling dilutions of the antibiotic. Observation for growth or no growth was performed after 16-20 hours of incubation at 35±2° C. in ambient air. The overall procedure was performed as per Clinical and Laboratory Standards Institute (CLSI) recommendations (Clinical and Laboratory Standards Institute (CLSI), performance Standards for Antimicrobial Susceptibility Testing, 20th Informational Supplement, M 100-S20, Volume 30, No. 1, 2010). The results of these studies are summarized in Tables 1-3.

Table 1 detail results of the activity study using a combination of amoxicillin+clavulanic acid and mecillinam against K. pneumoniae ATCC 700603 strain producing SHV 18 ESBL.

As seen from the data given in Table 1, each of amoxicillin and mecillinam have much higher MIC values indicating their lower antibiotic effectiveness, when used alone. A combination comprising amoxicillin+clavulanic acid and mecillinam+clavulanic acid showed moderate enhancement in the antibiotic effectiveness as compared when these were used alone. Surprisingly, the combination of amoxicillin+clavulanic acid+mecillinam exhibited substantially lower MIC value of 1 mc/gml.

TABLE 1
Antibacterial activity of amoxicillin in presence
of clavulanic acid and mecillinam against K. pneumoniae
ATCC 700603 strain producing SHV 18 ESBL

| | | MIC of Amoxicillin |
|---|---|---|
| Sr. | Composition | (mcg/ml) |
| 1. | Amoxicillin alone | >64 |
| 2. | Amoxicillin + Clavulanic acid (4 mcg/ml) | 8 |
| 3. | Amoxicillin + Mecillinam (4 mcg/ml) | >32 |
| 4. | Amoxicillin + Clavulanic acid (4 mcg/ml) + | 1 |
| | Mecillinam (4 mcg/ml) | |

MIC value for each of Clavulanic acid and Mecillinam when used alone was >64 mcg/ml.
MIC value for Mecillinam in combination with Clavulanic acid (4 mcg/ml) was 16 mcg/ml.

Table 2 provides data demonstrating surprising effect of the combination according to the invention on the killing of highly resistant ESBL producing pathogen (K. pneumoniae ATCC 700603 strain producing SHV 18 ESBL). As can be seen, bacterial cultures continued growth in control (drug free) and in the presence of drugs (for example, amoxicillin, clavulanic acid and mecillinam alone), indicating no antibacterial activity. In contrast, a combination comprising amoxicillin, clavulanic acid and mecillinam showed potent cidality bringing about 99 to 99.99% killing in 2 to 8 hours.

TABLE 2
Cidal activity of a combination comprising Amoxicillin,
Clavulanic acid and Mecillinam against K. pneumoniae
ATCC 700603 strain producing SHV 18 ESBL

| | | Bacterial count | | |
|---|---|---|---|---|
| | | (Log CFU/ml) | | |
| Sr. | Combination | 2 hours | 6 hours | 8 hours |
| 1. | Control (No active ingredient) | 7.90 | 9.14 | 9.3 |
| 2. | Amoxicillin (8 mcg/ml) | 7.84 | 8.5 | 9 |
| 3. | Clavulanic acid (4 mcg/ml) | 8.1 | 8.8 | 9 |
| 4. | Mecillinam (4 mcg/ml) | 7.47 | 8.23 | 9.1 |
| 5. | Amoxicillin (8 mcg/ml) + | 6.3 | 7.94 | 9 |
| | Clavulanic acid (4 mcg/ml) | | | |
| 6. | Amoxicillin (8 mcg/ml) + | 7.67 | 8.16 | 8.89 |
| | Mecillinam (4 mcg/ml) | | | |
| 7. | Mecillinam (4 mcg/ml) + | 6.65 | 6.1 | 5.91 |
| | Clavulanic acid (4 mcg/ml) | | | |
| 8. | Amoxicillin (4 mcg/ml) + | 4.02 | 2.95 | 2.1 |
| | Clavulanic acid (4 mcg/ml) + | | | |
| | Mecillinam (4 mcg/ml) | | | |
| 9. | Amoxicillin (8 mcg/ml) + | 3.77 | 2.2 | 1.6 |
| | Clavulanic acid (4 mcg/ml) + | | | |
| | Mecillinam (4 mcg/ml) | | | |
| 10. | Imipenem (2 mcg/ml) | 5.32 | 2.6 | 2.0 |

Initial bacterial count (at 0 hours) was 6.65 log CFU/ml

Table 3 provides data demonstrating surprising effect of the combination on the killing of another highly resistant ESBL producing pathogen (K. pneumoniae B 43 strain producing SHV and TEM ESBLs).

TABLE 3
Cidal activity of a combination comprising Amoxicillin,
Clavulanic acid and Mecillinam against K. pneumoniae
B 43 strain producing SHV and TEM ESBLs.

| | | Bacterial count | | |
|---|---|---|---|---|
| | | (Log CFU/ml) | | |
| Sr. | Combination | 2 hours | 6 hours | 8 hours |
| 1. | Control (No active ingredient) | 7.95 | 8.97 | 9 |
| 2. | Amoxicillin (8 mcg/ml) | 7.8 | 8.8 | 9 |
| 3. | Clavulanic acid (4 mcg/ml) | 8.1 | 8.7 | 9 |
| 4. | Mecillinam (4 mcg/ml) | 7.74 | 8.69 | 8.89 |
| 5. | Amoxicillin (8 mcg/ml) + | 8.16 | 8.60 | 9 |
| | Clavulanic acid (4 mcg/ml) | | | |
| 6. | Amoxicillin (8 mcg/ml) + | 7.57 | 8.49 | 8.90 |
| | Mecillinam (4 mcg/ml) | | | |
| 7. | Mecillinam (4 mcg/ml) + | 7.16 | 6.95 | 7.2 |
| | Clavulanic acid (4 mcg/ml) | | | |
| 8. | Amoxicillin (4 mcg/ml) + | 7.02 | 4.74 | 4.24 |
| | Clavulanic acid (4 mcg/ml) + | | | |
| | Mecillinam (4 mcg/ml) | | | |
| 9. | Amoxicillin (8 mcg/ml) + | 6.39 | 4.60 | 3.84 |
| | Clavulanic acid (4 mcg/ml) + | | | |
| | Mecillinam (4 mcg/ml) | | | |
| 10. | Imipenem (2 mcg/ml) | 5.4 | 3.9 | 3.8 |

Initial bacterial count (at 0 hours) was 7.04 log CFU/ml

Table 4 provides data demonstrating surprising effect of the combination (Cefixime+Clavulanic acid+Mecillinam) against K. pneumoniae ATCC 700603 strain producing SHV 18 ESBL. As can be seen, the combination (Cefixime+Clavulanic acid+Mecillinam) has pharmacodynamics to Carbapenem (for example, Imipenem).

TABLE 4
Cidal activity of a combination comprising Cefixime,
Clavulanic acid and Mecillinam against K. pneumoniae
ATCC 700603 strain producing SHV 18 ESBL

| | | Bacterial count | | |
|---|---|---|---|---|
| | | (Log CFU/ml) | | |
| Sr. | Combination | 2 hours | 4 hours | 8 hours |
| 1. | Control (No active ingredient) | 8.60 | 8.74 | 8.92 |
| 2. | Cefixime (2 mcg/ml) | 8.11 | 8.30 | 9 |
| 3. | Clavulanic acid (4 mcg/ml) | 8.4 | 8.65 | 8.9 |
| 4. | Mecillinam (2 mcg/ml) | 7.95 | 8.33 | 8.90 |
| 5. | Cefixime (1 mcg/ml) + | 6.47 | 5.60 | 3.13 |
| | Clavulanic acid (4 mcg/ml) | | | |
| 6. | Cefixime (2 mcg/ml) + | 6.09 | 4.39 | 3.10 |
| | Clavulanic acid (4 mcg/ml) | | | |
| 7. | Mecillinam (2 mcg/ml) + | 8.04 | 8.1 | 8.17 |
| | Clavulanic acid (4 mcg/ml) | | | |
| 8. | Cefixime (1 mcg/ml) + | 7.74 | 8.07 | 8.84 |
| | Mecillinam (2 mcg/ml) | | | |
| 9. | Cefixime (2 mcg/ml) + | 7.69 | 8.09 | 8.97 |
| | Mecillinam (2 mcg/ml) | | | |
| 10. | Cefixime (1 mcg/ml) + | 4.21 | 3.30 | 2.50 |
| | Clavulanic acid (4 mcg/ml) + | | | |
| | Mecillinam (2 mcg/ml) | | | |
| 11. | Cefixime (2 mcg/ml) + | 4.09 | 3 | 2.32 |
| | Clavulanic acid (4 mcg/ml) + | | | |
| | Mecillinam (2 mcg/ml) | | | |
| 12. | Imipenem (1 mcg/ml) | 4.87 | 3.39 | 2.84 |

Initial bacterial count (at 0 hours) was 7.43 log CFU/ml

Table 5 provides data demonstrating surprising effect of the combination (Cefixime+Clavulanic acid+Mecillinam) against K. pneumoniae B 43 strain producing SHV and TEM ESBLs. As can be seen, the combination (Cefixime+Clavulanic acid+Mecillinam) exhibits potent activity against ESBL producing strain causing 99% reduction in the count at the end of 8 hrs, where a carbapenem (for example, Imipenem) fails to kill consistently.

TABLE 5
Cidal activity of a combination comprising Cefixime,
Clavulanic acid and Mecillinam against K. pneumoniae
B 43 strain producing SHV and TEM ESBLs.

| | | Bacterial count | | |
|---|---|---|---|---|
| | | (Log CFU/ml) | | |
| Sr. | Combination | 2 hours | 4 hours | 8 hours |
| 1. | Control (No active ingredient) | 8.20 | 8.60 | 8.92 |
| 2. | Cefixime (2 mcg/ml) | 8.16 | 8.47 | 8.84 |
| 3. | Clavulanic acid (4 mcg/ml) | 8.1 | 8.56 | 8.85 |
| 4. | Mecillinam (2 mcg/ml) | 8.39 | 8.60 | 8.90 |
| 5. | Cefixime (1 mcg/ml) + | 7.54 | 5.92 | 6.47 |
| | Clavulanic acid (4 mcg/ml) | | | |
| 6. | Cefixime (2 mcg/ml) + | 7.60 | 7.60 | 5.77 |
| | Clavulanic acid (4 mcg/ml) | | | |
| 7. | Mecillinam (2 mcg/ml) + | 8.06 | 7.97 | 7.17 |
| | Clavulanic acid (4 mcg/ml) | | | |
| 8. | Cefixime (1 mcg/ml) + | 7.87 | 8.27 | 8.23 |
| | Mecillinam (2 mcg/ml) | | | |
| 9. | Cefixime (2 mcg/ml) + | 8.47 | 8.17 | 8.39 |
| | Mecillinam (2 mcg/ml) | | | |
| 10. | Cefixime (1 mcg/ml) + | 6.60 | 5.11 | 4.47 |
| | Clavulanic acid (4 mcg/ml) + | | | |
| | Mecillinam (2 mcg/ml) | | | |
| 11. | Cefixime (2 mcg/ml) + | 6.13 | 5.09 | 4.27 |
| | Clavulanic acid (4 mcg/ml) + | | | |
| | Mecillinam (2 mcg/ml) | | | |
| 12. | Imipenem (1 mcg/ml) | 6.37 | 5.69 | 6.60 |

Initial bacterial count (at 0 hours) was log 7.43 CFU/ml

Table 6 provides data demonstrating surprising effect of a combination (Cefpodoxime+Clavulanic acid+Mecillinam) against K. pneumoniae ATCC 700603 strain producing SHV 18 ESBL.

TABLE 6
Cidal activity of a combination comprising Cefpodoxime,
Clavulanic acid and Mecillinam against K. pneumoniae
ATCC 700603 strain producing SHV 18 ESBL

| | | Bacterial count | | |
|---|---|---|---|---|
| | | (Log CFU/ml) | | |
| Sr. | Combination | 2 hours | 4 hours | 8 hours |
| 1. | Control (No active ingredient) | 8.47 | 8.95 | 9.11 |
| 2. | Cefpodoxime (2 mcg/ml) | 8.54 | 9 | 9 |
| 3. | Clavulanic acid (4 mcg/ml) | 8.47 | 9.09 | 9.09 |
| 4. | Mecillinam (2 mcg/ml) | 8.23 | 8.74 | 9.47 |
| 5. | Cefpodoxime (1 mcg/ml) + | 6.60 | 5.60 | 6.54 |
| | Clavulanic acid (4 mcg/ml) | | | |
| 6. | Cefpodoxime (2 mcg/ml) + | 6.65 | 5.13 | 4.43 |
| | Clavulanic acid (4 mcg/ml) | | | |
| 7. | Mecillinam (2 mcg/ml) + | 7.77 | 8.26 | 8.25 |
| | Clavulanic acid (4 mcg/ml) | | | |
| 8. | Cefpodoxime (1 mcg/ml) + | 8.17 | 8.81 | 8.81 |
| | Mecillinam (2 mcg/ml) | | | |
| 9. | Cefpodoxime (2 mcg/ml) + | 8.02 | 8.84 | 9.02 |
| | Mecillinam (2 mcg/ml) | | | |
| 10. | Cefpodoxime (1 mcg/ml) + | 4.13 | 3.54 | 2.64 |
| | Clavulanic acid (4 mcg/ml) + | | | |
| | Mecillinam (2 mcg/ml) | | | |
| 11. | Cefpodoxime (2 mcg/ml) + | 4.19 | 3.47 | 2.72 |
| | Clavulanic acid (4 mcg/ml) + | | | |
| | Mecillinam (2 mcg/ml) | | | |
| 12. | Imipenem (1 mcg/ml) | 5.32 | 3.84 | 2.32 |

Initial bacterial count (at 0 hours) was 7.27 log CFU/ml

Table 7 provides data demonstrating surprising effect of a combination (Cefuroxime+Clavulanic acid+Mecillinam) against K. pneumoniae ATCC 700603 strain producing SHV 18 ESBL.

TABLE 7
Cidal activity of a combination comprising Cefuroxime,
Clavulanic acid and Mecillinam against K. pneumoniae
ATCC 700603 strain producing SHV 18 ESBL

| | | Bacterial count | | |
|---|---|---|---|---|
| | | (Log CFU/ml) | | |
| Sr. | Combination | 2 hours | 4 hours | 8 hours |
| 1. | Control (No active ingredient) | 8.47 | 8.95 | 9.11 |
| 2. | Cefuroxime (2 mcg/ml) | 8.77 | 8.90 | 9 |
| 3. | Clavulanic acid (4 mcg/ml) | 8.47 | 9.09 | 9.09 |
| 4. | Mecillinam (2 mcg/ml) | 8.23 | 8.74 | 9.47 |
| 5. | Cefuroxime (1 mcg/ml) + | 8.16 | 8.30 | 8.48 |
| | Clavulanic acid (4 mcg/ml) | | | |
| 6. | Cefuroxime (2 mcg/ml) + | 7.38 | 7.97 | 8.23 |
| | Clavulanic acid (4 mcg/ml) | | | |
| 7. | Mecillinam (2 mcg/ml) + | 7.77 | 8.26 | 8.25 |
| | Clavulanic acid (4 mcg/ml) | | | |
| 8. | Cefuroxime (1 mcg/ml) + | 8.09 | 8.54 | 8.51 |
| | Mecillinam (2 mcg/ml) | | | |
| 9. | Cefuroxime (2 mcg/ml) + | 7.90 | 8 | 8.44 |
| | Mecillinam (2 mcg/ml) | | | |
| 10. | Cefuroxime (1 mcg/ml) + | 7.04 | 4.92 | 4.30 |
| | Clavulanic acid (4 mcg/ml) + | | | |
| | Mecillinam (2 mcg/ml) | | | |
| 11. | Cefuroxime (2 mcg/ml) + | 5.90 | 4.16 | 3.28 |
| | Clavulanic acid (4 mcg/ml) + | | | |
| | Mecillinam (2 mcg/ml) | | | |
| 12. | Imipenem (1 mcg/ml) | 5.32 | 3.84 | 2.32 |

Initial bacterial count (at 0 hours) was 7.27 log CFU/ml

The data provided in Tables 1 to 7, indicate that a combination comprising at least one beta-lactam antibiotic agent, at least one beta-lactamase inhibitor and mecillinam or a pharmaceutically acceptable derivative can be effectively used in treating or controlling bacterial infections (even those being caused by bacteria producing one or more beta-lactamase enzymes) in a subject.

1. A pharmaceutical composition useful for the treatment or control of bacterial infections, the composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

2. A method for the treatment or control of bacterial infections, said method comprising administering to a patient in need thereof, effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

3. A method for the treatment or control of bacterial infections, said method comprising administering to a patient in need thereof, a pharmaceutical composition comprising effective amounts of: (a) at least one beta-lactam antibiotic agent or a pharmaceutically acceptable derivative thereof, (b) at least one beta-lactamase inhibitor or a pharmaceutically acceptable derivative thereof, and (c) mecillinam or a pharmaceutically acceptable derivative thereof, wherein the beta-lactam antibiotic agent is not mecillinam or a pharmaceutically acceptable derivative thereof.

4. A method for increasing antibiotic effectiveness of a beta-lactam antibiotic agent in a subject, said method comprising co-administering said beta-lactam antibiotic agent with effective amounts of: (a) at least one beta-lactamase inhibitor or a pharmaceutically acceptable derivative thereof, and (b) mecillinam or a pharmaceutically acceptable derivative thereof, wherein the beta-lactam antibiotic agent is not mecillinam or a pharmaceutically acceptable derivative thereof.

5. A pharmaceutical composition according to claim 1 or a method according to any of the claims 2 to 4, wherein the beta-lactam antibiotic agent is selected from the group consisting of penicillins, penems, carbapenems, cephems, and monobactams.

6. A pharmaceutical composition according to claim 1 or a method according to claims 2 to 4, wherein the beta-lactam antibiotic is selected from amoxicillin, ampicillin, pivampicillin, hetacillin, bacampicillin, metampicillin, talampicillin, epicillin, carbenicillin (carindacillin), ticarcillin, temocillin, azlocillin, piperacillin, mezlocillin, sulbenicillin, benzylpenicillin (G), clometocillin, benzathine benzylpenicillin, azidocillin, penamecillin, phenoxymethylpenicillin (V), propicillin, benzathine phenoxymethylpenicillin, pheneticillin, cloxacillin, dicloxacillin, flucloxacillin, oxacillin, meticillin, nafcillin, faropenem, biapenem, ertapenem, doripenem, imipenem, meropenem, panipenem, cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cefoxitin, cefotetan, cefmetazole, loracarbef, cefixime, ceftriaxone, ceftazidime, cefoperazone, cefcapene, cefdaloxime, cefdinir, cefditoren, cefetamet, cefmenoxime, cefodizime, cefotaxime, cefpimizole, cefpiramide, cefpodoxime, cefsulodin, cefteram, ceftibuten, ceftiolene, ceftizoxime, flomoxef, latamoxef, cefepime, cefozopran, cefpirome, cefquinome, ceftobiprole, ceftaroline fosamil, ceftiofur, cefquinome, cefovecin, aztreonam, tigemonam, carumonam, nocardicin A.

7. A pharmaceutical composition according to claim 1 or a method according to claims 2 to 4, wherein the beta-lactam antibiotic is selected from amoxicillin, cefixime, cefuroxime, cefpodoxime, cefaclor, cefprozil, cefdinir, cefditoren or a pharmaceutically acceptable derivative thereof.

8. A pharmaceutical composition according to claim 1 or a method according to claims 2 to 4, wherein the beta-lactamase inhibitor is selected from sulbactam, tazobactam, clavulanic acid, or a pharmaceutically acceptable derivative thereof.

9. A pharmaceutical composition according to claim 1, wherein beta-lactam antibiotic agent or a pharmaceutically acceptable derivative thereof is present in an amount of about 0.1 mg to about 3000 mg.

10. A pharmaceutical composition according to claim 1, wherein the beta-lactamase inhibitor or a pharmaceutically acceptable derivative thereof is present in an amount of about 0.1 mg to about 3000 mg.

11. A pharmaceutical composition according to claim 1, wherein mecillinam or a pharmaceutically acceptable derivative thereof is present in an amount of about 0.1 mg to about 3000 mg.

12. A pharmaceutical composition according to claim 1, wherein the composition comprising effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

13. A method according to claim 2, wherein the method comprising administering to a patient in need thereof, effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

14. A method according to claim 3, wherein the method comprising administering to a patient in need thereof, a pharmaceutical composition comprising effective amounts of: (a) amoxicillin or a pharmaceutically acceptable derivative thereof, (b) clavulanic acid or a pharmaceutically acceptable derivative thereof, and (c) mecillinam or a pharmaceutically acceptable derivative thereof.

15. A pharmaceutical composition according to claim 1, wherein the composition comprising effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

16. A method according to claim 2, wherein the method comprising administering to a patient in need thereof, effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

17. A method according to claim 3, wherein the method comprising administering to a patient in need thereof, a pharmaceutical composition comprising effective amounts of: (a) cefixime or a pharmaceutically acceptable derivative thereof, (b) clavulanic acid or a pharmaceutically acceptable derivative thereof, and (c) mecillinam or a pharmaceutically acceptable derivative thereof.

18. A pharmaceutical composition according to claim 1, wherein the composition comprising effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

19. A method according to claim 2, wherein the method comprising administering to a patient in need thereof, effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

20. A method according to claim 3, wherein the method comprising administering to a patient in need thereof, a pharmaceutical composition comprising effective amounts of: (a) cefuroxime or a pharmaceutically acceptable derivative thereof, (b) clavulanic acid or a pharmaceutically acceptable derivative thereof, and (c) mecillinam or a pharmaceutically acceptable derivative thereof.

21. A pharmaceutical composition according to claim 1, wherein the composition comprising effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.

22. A method according to claim 2, wherein the method comprising administering to a patient in need thereof, effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.

23. A method according to claim 3, wherein the method comprising administering to a patient in need thereof, a pharmaceutical composition comprising effective amounts of: (a) cefpodoxime or a pharmaceutically acceptable derivative thereof, (b) clavulanic acid or a pharmaceutically acceptable derivative thereof, and (c) mecillinam or a pharmaceutically acceptable derivative thereof.
